(19) United States
(12) Patent Application Publication
(10) Pub. No.: US 2023/0329980 A1
(43) Pub. Date: Oct. 19, 2023

(54) SOLID COSMETIC

(71) Applicant: Ocean View Holdings, LLC (dba Dew Mighty), Hermosa Beach, CA (US)

(72) Inventor: Tiffany Joy Buzzatto, Hermosa Beach, CA (US)

(73) Assignee: Ocean View Holdings, LLC (dba Dew Mighty), Hermosa Beach, CA (US)

(21) Appl. No.: 18/043,619

(22) PCT Filed: Aug. 26, 2021
(86) PCT No.: PCT/US2021/047720

§ 371 Date: Mar. 1, 2023

Related U.S. Application Data
(60) Provisional application No. 63/073,237, filed on Sep. 1, 2020.

(51) Int. Cl. A61K 8/02; A61K 8/37; A61K 8/42; A61K 8/92; A61K 8/9789; A61Q 19/00

(52) U.S. Cl. CPC A61K 8/0216; A61K 8/37; A61K 8/42; A61K 8/922; A61K 8/9789; A61Q 19/00; A61K 2800/31; A61K 2800/30; A61K 2800/262

(57) ABSTRACT

A solid cosmetic, methods of making the same, packaged products including the same, and refillable skin care systems including the same. A solid cosmetic includes an amino acid oil-gelling agent and a fatty acid ester component that includes a (C5-C15)alkyl ester of a (C14-C20) fatty acid. The solid cosmetic is substantially free of water and polyamides.

### CROSS-REFERENCE TO RELATED APPLICATION

This application claims the benefit of priority to U.S. Provisional Patent Application Ser. No. 63/073,237 filed Sep. 1, 2020, the disclosure of which is incorporated herein in its entirety by reference.

### BACKGROUND

Conventional cosmetics include an emulsified system combining an aqueous phase and an oily phase, which allow a combination of cosmetic active agents having different aqueous/oil affinities within the same system. However, these emulsified systems can be inconvenient because variation of the type or amount of materials in the emulsion can lead to destabilization of the emulsion and consequent variation of both the visual properties of the composition as well as the topical performance of the composition. In addition, the water adds unnecessary volume to the composition, raising transportation costs for the product. Anhydrous cosmetics are generally in the form of liquids or solids that require components that make the product opaque, readily water-soluble, and non-resistant to melting.

### SUMMARY OF TUE INVENTION

A solid cosmetic includes an amino acid oil-gelling agent. The solid cosmetic also includes a fatty acid ester component including a (C₅-C₁₅)alkyl ester of a (C₁₄-C₂₀)fatty acid. The solid cosmetic is substantially free of water and polyamides.

A solid cosmetic includes an amino acid oil-gelling agent including dibutyl lauroyl glutamide, dibutyl ethylhexanoyl glutamide, or a combination thereof, wherein the amino acid oil-gelling agent is 1 wt % to 30 wt % of the solid cosmetic. The solid cosmetic includes a fatty acid ester component including a (C₅-C₁₅)alkyl ester of a (C₁₆)fatty acid and a (C₅-C₁₅)alkyl ester of a (C₁₈)fatty acid, wherein the fatty acid ester component is 20 wt % to 60 wt % of the solid cosmetic. The solid cosmetic also includes one or more seed or vegetable oils that are 20 wt % to 60 wt % of the solid cosmetic. Water and polyamides are less than 0.01 wt % of the solid cosmetic.

A solid cosmetic includes an amino acid oil-gelling agent including dibutyl lauroyl glutamide and dibutyl ethylhexanoyl glutamide having a weight ratio of 1:1 to 5:1, wherein the amino acid oil-gelling agent is 5 wt % to 15 wt % of the solid cosmetic. The solid cosmetic includes a fatty acid ester component including ethylhexyl olivate, wherein the fatty acid ester component is 35 wt % to 45 wt % of the solid cosmetic. The solid cosmetic also includes one or more seed or vegetable oils including Jojoba oil, wherein the Jojoba oil is 30 wt % to 50 wt % of the solid cosmetic. Water and polyamides are less than 0.01 wt % of the solid cosmetic.

A solid cosmetic includes an amino acid oil-gelling agent including dibutyl lauroyl glutamide, dibutyl ethylhexanoyl glutamide, or a combination thereof, wherein the amino acid oil-gelling agent is 1 wt % to 30 wt % of the solid cosmetic. The solid cosmetic includes a fatty acid ester component including a (C₅-C₁₅)alkyl ester of a (C₁₆)fatty acid and a (C₅-C₁₅)alkyl ester of a (C₁₈)fatty acid, wherein the fatty acid ester component is 20 wt % to 60 wt % of the solid cosmetic. The solid cosmetic includes one or more seed or vegetable oils that are 20 wt % to 60 wt % of the solid cosmetic. The solid cosmetic includes a phase B component including an antioxidant, a plant oil, an evergreen flower extract, a Hibiscus seed oil, a licorice extract or derivative thereof, a filler, a flowering tree bark extract, or a combination thereof, wherein the phase B component is 1 wt % to 30 wt % of the solid cosmetic. The solid cosmetic also includes a phase C component including a tansy oil, an Annonaceae flower oil, an Oleaceae flower oil, a chamomile extract, or a combination thereof, wherein the phase C component is 0.001 wt % to 5 wt % of the solid cosmetic. Water and polyamides are less than 0.01 wt % of the solid cosmetic.

A solid cosmetic includes an amino acid oil-gelling agent including dibutyl lauroyl glutamide, dibutyl ethylhexanoyl glutamide, or a combination thereof, wherein the amino acid oil-gelling agent is 1 wt % to 30 wt % of the solid cosmetic. The solid cosmetic includes a fatty acid ester component including a (C₅-C₁₅)alkyl ester of a (C₁₆)fatty acid and a (C₅-C₁₅)alkyl ester of a (C₁₈)fatty acid, wherein the fatty acid ester component is 20 wt % to 60 wt % of the solid cosmetic. The solid cosmetic includes one or more seed or vegetable oils that are 20 wt % to 60 wt % of the solid cosmetic. The solid cosmetic includes a phase B component including an antioxidant, a plant oil, an evergreen flower extract, a Hibiscus seed oil, a licorice extract or derivative thereof, a filler, and a flowering tree bark extract, wherein the phase B component is 1 wt % to 30 wt % of the solid cosmetic. The solid cosmetic also includes a phase C component including a tansy oil; an Annonaceae flower oil, an Oleaceae flower oil, and a chamomile extract, wherein the phase C component is 0.001 wt % to 5 wt % of the solid cosmetic. Water and polyamides are less than 0.01 wt % of the solid cosmetic.

A solid cosmetic includes an amino acid oil-gelling agent including dibutyl lauroyl glutamide and dibutyl ethylhexanoyl glutamide having a weight ratio of 1:1 to 5:1, wherein the amino acid oil-gelling agent is 5 wt % to 15 wt % of the solid cosmetic. The solid cosmetic includes a fatty acid ester component including ethylhexyl olivate, wherein the fatty acid ester component is 35 wt % to 45 wt % of the solid cosmetic. The solid cosmetic includes one or more seed or vegetable oils including Jojoba oil, wherein the Jojoba oil is 30 wt % to 50 wt % of the solid cosmetic. The solid cosmetic includes a phase B component including tetrahexyldecyl ascorbate, Cocos Nucifera oil, Gardenia Tahitensis flowers extract, Hibiscus sabdariffa seed oil, stearyl glycyrrhetinate, a silica, and Magnolia officinalis bark extract, wherein the phase B component is 5 wt % to 15 wt % of the solid cosmetic. The solid cosmetic also includes a phase C component including Tanacetum annum essential oil, Cananga Odorata Genuina flower extract, Jasmine oil, and Chamomilla recutita extract, wherein the phase C component is 0.01 wt % to 1 wt % of the solid cosmetic. Water and polyamides are less than 0.01 wt % of the solid cosmetic.

A method of making a solid cosmetic includes combining the amino acid oil-gelling agent and one or more oils including the fatty acid ester component including a (C₅-C₁₅)alkyl ester of a (C₁₄-C₂₀)fatty acid to form a mixture. The method includes heating the mixture to a temperature within and not exceeding a first temperature range of 145-160° C. sufficiently to substantially dissolve the amino acid oil-gelling agent in the one or more oils. The method includes adding the phase B component including an antioxidant, a plant oil, an evergreen flower extract, a Hibiscus seed oil, a licorice extract or derivative thereof, a filler, a flowering tree bark extract, or a combination thereof to the mixture and heating the mixture within and not exceeding the first temperature range sufficiently to achieve a translucent mixture. The method also includes transferring the translucent mixture to molds while the translucent mixture is within and not less than a second temperature range of 140-155° C., to form the solid cosmetic.

A method of making a solid cosmetic includes combining the amino acid oil-gelling agent and one or more oils including the fatty acid ester component including a (C₅-C₁₅)alkyl ester of a (C₁₄-C₂₀fatty acid to form a mixture. The method includes heating the mixture to a temperature within and not exceeding a first temperature range of 145-160° C. sufficiently to substantially dissolve the amino acid oil-gelling agent in the one or more oils. The method includes adding the phase B component including an antioxidant, a plant oil, an evergreen flower extract, a Hibiscus seed oil, a licorice extract or derivative thereof, a filler, a flowering tree bark extract, or a combination thereof to the mixture and heating the mixture within and not exceeding the first temperature range sufficiently to achieve a translucent mixture. The method includes heating the phase C component including a tansy oil, an Annonaceae flower oil, an Oleaceae flower oil, a chamomile extract, or a combination thereof to within a third temperature range less than the second temperature range, wherein the third temperature range is 40-60° C. The method includes adding the phase C component to the translucent mixture. The method includes heating the mixture to within and not exceeding the second temperature range while agitating the mixture until it is again translucent. The method also includes transferring the translucent mixture to molds while the translucent mixture is within and not less than a second temperature range of 140-155° C. to form the solid cosmetic. The transferring of the translucent mixture to the molds while the translucent mixture is within and not less than a second temperature range includes transferring the translucent mixture to the molds without any intermediary steps of storage, cooling, and reheating. The transferring of the mixture to the molds is completed within 0.01-20 minutes of the achieving of the translucent mixture after the addition of the phase C component.

A method of making a solid cosmetic includes combining the amino acid oil-gelling agent including dibutyl lauroyl glutamide and dibutyl ethylhexanoyl glutamide having a weight ratio of 1:1 to 5:1, one or more oils including ethylhexyl olivate, and one or more seed or vegetable oils including Jojoba oil, to form a mixture. The method includes heating the mixture to a temperature within and not exceeding a first temperature range of 154-156° C. sufficiently to substantially dissolve the amino acid oil-gelling agent in the one or more oils. The method includes adding the phase B component including tetrahexyldecyl ascorbate, Cocos Nucifera oil, Gardenia Tahitensis flowers extract, Hibiscus sabdariffa seed oil, stearyl glycyrrhetinate, a silica, and Magnolia officinalis bark extract to the mixture and heating the mixture within and not exceeding the first temperature range sufficiently to achieve a translucent mixture. The method includes heating the phase C component including a Tanacetum annum essential oil, Cananga Odorata Genuina flower extract, Jasmine oil, and Chamomilla recutita extract to within a third temperature range less than the second temperature range, wherein the third temperature range is 45-55° C. The method includes adding the phase C component to the translucent mixture. The method includes heating the mixture to within and not exceeding the second temperature range while agitating the mixture until it is again translucent. The method also includes transferring the translucent mixture to molds while the translucent mixture is within and not less than a second temperature range of 147-149° C., to form the solid cosmetic. The transferring of the translucent mixture to the molds while the translucent mixture is within and not less than a second temperature range includes transferring the translucent mixture to the molds without any intermediary steps of storage, cooling below the second temperature range, and reheating. The transferring of the mixture to the molds is completed within 0.01-10 minutes of the achieving of the translucent mixture after the addition of the phase C component. The amino acid oil-gelling agent is 5 wt % to 15 wt % of the solid cosmetic. The fatty acid ester component is 35 wt % to 45 wt % of the solid cosmetic. The Jojoba oil is 30 wt % to 50 wt % of the solid cosmetic. The phase B component is 5 wt % to 15 wt % of the solid cosmetic. The phase C component is 0.01 wt % to 1 wt % of the solid cosmetic.

A packaged product includes the solid cosmetic. The packaged product also includes packaging that is resistant to the absorption of oils from the solid cosmetic.

A refillable skin care system includes the solid cosmetic. The refillable skin care system also includes a refillable container for storing the solid cosmetic.

In various aspects, the solid cosmetic of the present invention and method of making the same have certain advantages over other skin care products and methods of making the same. For example, in various aspects, the solid cosmetic of the present invention is easy to apply, transferring via friction and contact with an absorbent surface (e.g., skin or hair). In various aspects, the solid cosmetic of the present invention has good storage stability in a wide range of temperatures (e.g., will not melt and/or degrade during transport/storage or prior to/during use). In various aspects, variation of the composition of the solid cosmetic of the present invention with maintenance or variation of performance is facile. In various aspects, the solid cosmetic of the present invention is visually appealing, as a translucent skin bar. In various aspects, the solid cosmetic of the present invention provides a more concentrated product that is not diluted with water (unlike emulsified systems) and thereby saves on shipping costs, storage space, and provides a product that provides a greater number of uses that a similar volume of emulsified skin care product.

In various aspects, the method of making the solid cosmetic of the present invention reduces and/or or avoids degradation of temperature-sensitive components, thereby providing a visually satisfying translucent solid cosmetic including undegraded natural ingredients that the consumer can enjoy at their full undegraded potency levels or with decreased degradation as compared to some other products containing the natural ingredient.

In various aspects, the packaged solid cosmetic of the present invention includes packaging that is resistant to getting soaked with oils from the solid cosmetic, with the packaging providing easy transportation, storage, and protection of the solid cosmetic.

### DETAILED DESCRIPTION OF THE INVENTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B,” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%. 70%, 80%, 90%, 95%, 96%, 97%. 98%, 99%, 99.5%, 99.9%, 99,99%, or at least about 99.999% or more, or 100%. The term “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than, equal to, or greater than about 4.5 wt %, 4, 3,5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl.

### Solid Cosmetic

Various aspects of the present invention provide a solid cosmetic. The solid cosmetic includes an amino acid oil-gelling agent. The solid cosmetic also includes a fatty acid ester component including a (C₅-C₁₅)alkyl ester of a (C₁₄-C₂₀)fatty acid. The solid cosmetic is substantially free of water and polyamides.

The solid cosmetic can have any suitable shape, such as a puck or a bar. The solid cosmetic can be an oily solid cosmetic and/or an oleaginous solid cosmetic. The solid cosmetic is a firm oily solid at room temperature, which transfers the components thereof to the skin upon friction therewith. The solid cosmetic can be transparent or translucent, and can be free of suspended particles as measured by inspection with the naked eye. Light can readily pass through the solid cosmetic upon inspection with the naked eye. The solid cosmetic can have any suitable color, which can depend on the components used to form the solid cosmetic.

The solid cosmetic can be free of synthetic dyes. For example, less than 0.01 wt % of the solid cosmetic can be synthetic dyes, or less than 0.001 wt %, or less than 0.0001 wt %.

The solid cosmetic can be free of synthetic fragrances. For example, less than 0.01 wt % of the solid cosmetic can be synthetic fragrances, or less than 0.001 wt %, or less than 0.0001 wt %.

The solid cosmetic can be free of water. For example, less than 0.01 wt % of the solid cosmetic can be water, or less than 0.001 wt %, or less than 0.0001 wt %.

The solid cosmetic can be free of polyamides. For example, less than 0.01 wt % of the solid cosmetic can be polyamides, or less than 0.001 wt %, or less than 0.0001 wt %.

The solid cosmetic includes a fatty acid ester component including a (C₅-C₁₅)alkyl ester of a (C₁₄-C₂₀)fatty acid. The (C₅-C₁₅)alkyl of the (C₁₄-C₂₀)fatty acid can be branched or unbranched. The (C₅-C₁₅)alkyl of the (C₁₄-C₂₀)fatty acid can be branched. The (C₅-C₁₅)alkyl ester of the (C₁₄-C₂₀)fatty acid can be a (C₇-C₁₀)alkyl ester. The (C₅-C₁₅)alkyl ester of the (C₁₄-C₂₀)fatty acid can be a (C₈)alkyl ester. The (C₅-C₁₅)alkyl ester of the (C₁₄-C₂₀)fatty acid can be ethylhexyl. The (C₁₄-C₂₀)fatty acid can be a (C₁₆-C₁₈)fatty acid. The (C₁₄-C₂₀)fatty acid can be or include a (C₁₆)fatty acid. The (C₁₄-C₂₀)fatty acid can be or include a (C₁₈)fatty acid. The fatty acid ester component can include a (C₅-C₁₅)alkyl ester of a (C₁₆)fatty acid and a (C₅-C₁₅)alkyl ester of a (C₁₈)fatty acid. The fatty acid ester component can include ethylhexyl hexadecanoic acid (e.g., from palmitic acid having C16:0), ethylhexyl octadecenoic acid (e.g., from oleic acid having C18:1), ethylhexyl linoleate (e.g., from linoleic acid having C18:2), or a combination thereof The fatty acid ester component can be 20 wt % to 60 wt % of the solid cosmetic, or 35 wt % to 45 wt %, or 20 wt % or more, or less than or equal to 60 wt % but greater than or equal to 22 wt %, 24, 26, 28, 30, 32, 34, 35, 36, 37, 38, 39, 40, 41, 42, 43, 44, 45, 46, 48, 50, 52, 54, 56, or 58 wt %.

The fatty acid ester component can include or can be ethylhexyl olivate. Ethylhexyl olivate is an olive oil-derived ester that can impart beneficial fatty acids, sterols, polyphenols and naturally occurring vitamins (E & K) to the skin. Olive oil contains primarily oleic acid, palmitic acid, and linoleic acid. The ethylhexyl olivate can be 20 wt % to 60 wt % of the solid cosmetic, or 35 wt % to 45 wt %, or less than or equal to 60 wt % but greater than or equal to 22 wt %, 24, 26, 28, 30, 32, 34, 35, 36, 37, 38. 39, 40, 41, 42, 43, 44, 45, 46, 48, 50, 52. 54, 56, or 58 wt %.

The solid cosmetic includes an amino acid oil-gelling- agent. The amino acid oil-gelling agent can be 1 wt % to 30 wt % of the solid cosmetic, or 5 wt % to 15 wt %, or less than or equal to 30 wt % but greater than or equal to 2 wt %, 3. 4. 5, 6, 7, 8, 9, 10, 11, 12, 13, 14, 15, 16, 17, 18, 19, 20, 21, 22, 23, 24, 25, 26, 27, 28, or 29 wt % of the solid cosmetic. The amino acid oil-gelling agent can be any suitable amino acid oil-gelling agent. For example, the amino acid oil-gelling agent can include dibutyl lauroyl glutamide, dibutyl ethylhexanoyl glutamide, or a combination thereof. For example, dibutyl lauroyl glutamide can be 1 wt % to 30 wt % of the solid cosmetic, or 2 wt % to 12 wt %, or less than or equal to 30 wt % but equal to or greater than 2 wt %, 3, 4, 5, 6, 7, 8, 9, 10, 11, 12, 13, 14, 15, 16, 17, 18, 19, 20, 21, 22, 23, 24, 25, 26, 27, 28, or 29 wt % of the solid cosmetic. Dibutyl ethylhexanoyl glutamide can be 1 wt % to 30 wt % of the solid cosmetic, or 0.1 wt % to 10 wt %, or 1 wt % to 5 wt % of the solid cosmetic, or less than or equal to 30 wt % but greater than or equal to 2 wt %, 2.2, 2.4, 2.6, 2.8. 3, 3.2, 3.4, 3.6, 3.8, 4, 5, 6, 7, 8, 9, 10, 11, 12, 13, 14, 15, 16, 17, 18, 19, 20, 21., 22, 23, 24, 25, 26, 27, 28, or 29 wt % of the solid cosmetic. The solid cosmetic can include both dibutyl lauroyl glutamide and dibutyl ethylhexanoyl glutamide, and the weight ratio of the dibutyl lauroyl glutamide to the dibutyl ethylhexanoyl glutamide can be 1:10 to 10:1, or 1:1 to 5:1, or less than or equal to 10:1 but equal to or greater than 1:10, 1:9, 1:8, 1:7, 1:6, 1:5, 1:4, 1:3, 1:2, 1:1, 2:1, 3:1, 4:1, 5:1, 6:1, 7:1, 8:1, or 9:1.

The solid cosmetic can further include one or more seed or vegetable oils. The one or more seed or vegetable oils, the amino acid oil-gelling agent, and the fatty acid ester component, can together form 70 wt % to 99 wt % of the solid cosmetic, or 85 wt % to 95 wt % of the solid cosmetic, or greater than or equal to 70 wt %, 72, 74, 76, 78, 80, 82, 84, 85, 86, 87, 88, 89, 90, 91, 92, 93, 94, 95, 96, 98, or 99 wt % or more. The one or more seed or vegetable oils can be 20 wt % to 60 wt % of the solid cosmetic, or 30 wt % to 50 wt %, or less than or equal to 60 wt % but equal to or greater than 20 wt %, 22, 24, 26, 28, 30, 31, 32, 33, 34, 35, 36, 37, 38, 39, 40, 41, 42, 43, 44, 45, 46, 47, 48, 49, 50, 52, 54, 56, or 58 wt %. The one or more seed or vegetable oils can include coconut oil, corn oil, canola oil, cottonseed oil, olive oil, palm oil, peanut oil, rapeseed oil, safflower oil, sesame oil, soybean oil, sunflower oil, cocoa butter, shea butter, mango butter, Jojoba oil, a fraction thereof, a middle or high oleic version thereof, a hydrogenated oil formed therefrom, an esterified or interesterified oil formed therefrom, or a combination thereof.

The one or more seed or vegetable oils can include or can be Jojoba oil. Jojoba oil is the liquid produced in the seed of the Simmondsia chinensis (jojoba) plant, a shrub, which is native to southern Arizona, southern California, and northwestern Mexico. The Jojoba oil can be cold pressed organic oil from jojoba seeds. The fatty acids can complement the skin's naturally composition to replenish and hydrate. The jojoba oil can be 20 wt % to 60 wt % of the solid cosmetic, or 30 wt % to 50 wt %, or less than or equal to 60 wt % but equal to or greater than 20 wt %, 22, 24, 26, 28, 30, 31, 32, 33, 34, 35, 36, 37, 38, 39, 40, 41, 42, 43, 44, 45, 46, 47, 48, 49, 50, 52, 54, 56, or 58 wt %.

The solid cosmetic can include a triterpene. Triterpenes are a class of chemical compounds composed of three terpene units with the molecular formula C₃₀H₄₈; they may also be thought of as consisting of six isoprene units. Animals, plants and fungi all produce triterpenes, including squalene, the precursor to all steroids. The triterpene can include squalene, and/or can include a ring skeleton corresponding to polypodatetraene, malabaricane, lanostane, cucurbitacin, hopane, oleanane, ursolic acid, or a combination thereof. The triterpene can include or be squalene. The triterpene can be 0.1 wt % to 10 wt % of the solid cosmetic, or 1 wt % to 5 wt %, or less than or equal to 10 wt % but greater than or equal to 0.1 wt %, 0.5, 1, 1.5, 2, 2.5, 3, 3.5, 4, 4.5, 5, 6, 7, 8, or 9 wt %.

The amino acid oil-gelling agent, the fatty acid ester component including a (C₅-C₁₅)alkyl ester of a (C₁₄-C₂₀)fatty acid, the one or more seed or vegetable oils, and the triterpene, can be referred to as phase A of the solid cosmetic. The term “phase” is not intended to mean that these components form a separate phase within the solid cosmetic that is not fully miscible with the other phases, but rather is merely used to refer to the primary combination of components of the solid cosmetic that together form the base for the other components in the solid cosmetic (e.g., phases B and C). In the solid cosmetic, phases A, B, and C are miscible with one another and form a homogeneous composition.

In the solid cosmetic, the amino acid oil-gelling agent can react with one or more other components (e.g., with phase A components, such as with the fatty acid ester component including a (C₅-C₁₅)alkyl ester of a (C₁₄-C₂₀)fatty acid, one or more seed or vegetable oils, the triterpene, one or more other components, or a combination thereof) to form the solid structure of the solid cosmetic (i.e., to form a gel that holds and stabilizes the oils and other ingredients for transport, storage, and skin application). For any embodiment of the solid cosmetic described herein, at least some proportion of the amino acid-oil gelling agent can be reacted with one or more other components, such as 0.01 wt % to 100 wt % of the amino acid-oil gelling agent, or less than, equal to, or greater than 0.01 wt %, 0.1, 1, 2, 3, 4, 5, 10, 15, 20, 25, 30, 35, 40, 45, 50, 55, 60, 65, 70, 75, 80, 85, 90, 95, 96, 97, 98, 99, 99.9, or 99.99 wt % or more.

The solid cosmetic can include a phase B component. The phase B component can include one or more components. The phase B component can be 1 wt % to 30 wt % of the solid cosmetic, or 5 wt % to 15 wt % of the solid cosmetic, or less than or equal to 30 wt % but greater than or equal to 1 wt %, 2, 3, 4, 5, 6, 7, 8, 9, 10, 11, 12, 13 14, 15, 16, 18, 20, 22, 24, 26, or 28 wt %. The phase B component can include any one or more suitable plant-based components. The phase B component can include an antioxidant, a plant oil, an evergreen flower extract, a Hibiscus seed oil, a licorice extract or derivative thereof, a filler, a flowering tree bark extract, or a combination thereof.

The phase B component can include one or more antioxidants. The one or more antioxidants can include ascorbic acid, glutathione, lipoic acid, uric acid, a carotene, alpha tocopherol, ubiquinol, a derivative thereof, or a combination thereof. The one or more antioxidants can be or include tetrahexyldecyl ascorbate. Tetrahexyldecyl ascorbate is a stabilized vitamin C that imparts benefits without threat of prior oxidation. It has the ability to even skin tone appearance, smooth skin appearance, and act as a powerful anti-oxidant. The one or more antioxidants can be 0.01 wt % to 10 wt % of the solid cosmetic, or 0.1 wt % to 5 wt %, or less than or equal to 10 wt % but greater than or equal to 0.01 wt %, 0.1, 0.5. 0.8, 1.0, 1.2, 1.4, 1.6, 1.8, 2, 3, 4, 5, 6, 7, 8, or 9 wt %.

The phase B component can include one or more plant oils. The one or more plant oils can include coconut oil, corn oil, canola oil, cottonseed oil, olive oil, palm oil, peanut oil, rapeseed oil, safflower oil, sesame oil, soybean oil, sunflower oil, cocoa butter, shea butter, mango butter, Jojoba oil, a fraction thereof, a middle or high oleic version thereof, a hydrogenated oil formed therefrom, an esterified or interesterified oil formed therefrom, or a combination thereof. The one or more plant oils can include or be Cocos Nucifera oil (i.e. coconut oil). The one or more plant oils can be 1 wt % to 20 wt % of the solid cosmetic, or 5 wt % to 10 wt %, or less than or equal to 20 wt % but equal to or greater than 1 wt %, 2, 3, 4, 5, 6, 7, 8, 9, 10, 11, 12, 13, 14, 15, 16, 17, 18, or 19 wt % of the solid cosmetic.

The phase B component can include one more evergreen flower extracts. An evergreen is a plant that has leaves throughout the year and are always green. This is true even if the plant retains its foliage only in warm climates, and contrasts with deciduous plants, which completely lose their foliage during the winter or dry season. The one or more evergreen flower extracts can include Gardenia Tahitensis flowers extract. The Gardenia Tahitensis flower extract can be an extract of the Gardenia Thitensis flower that has been extracted using coconut oil. The The fragrant extract of the Gardenia Thitensis flower is highly touted for its beautifying properties, The one or more evergreen flower extracts can be 0.0001 wt % to 5 wt % of the solid cosmetic, or 0.001 wt % to 1 wt %, or less than or equal to 5 wt % but greater than or equal to 0.0001 wt %, 0.0005, 0.001, 0.005, 0.01, 0.05, 0.1, 0.5, 1, 2, 3, or 4 wt %.

The phase B component can include a Hibiscus seed oil. Hibiscus is a genus of flowering plants in the mallow family, Malvaceae. The genus is quite large, comprising several hundred species that are native to warm temperate, subtropical and tropical regions throughout the world. Member species are renowned for their large, showy flowers and those species are commonly known simply as “Hibiscus”, or less widely known as rose mallow. Other names include hardy Hibiscus, rose of sharon, and tropical Hibiscus. The Hibiscus seed oil can include an oil of Hibiscus abelmoschus L., Hibiscus abelmoschus var. betulifolius Mast., Hibiscus abelmoschus var. genuinus Hochr., Hibiscus laevis abutiloides Willd., Hibiscus abyssinicus Steud., Hibiscus acapulcensis Fryxell, Hibiscus acerifolius Salisb., Hibiscus acerifolius DC., Hibiscus acetosaefolius DC., Hibiscus acetosella Welw. ex Hiern., Hibiscus acicularis, Hibiscus aculeates, Hibiscus latissimus, Hibiscus andongensis, Hibiscus angolensis, Hibiscus aponeurus, Hibiscus archeri, Hibiscus aridicola, Hibiscus arnottianus A. Gray, Hibiscus asper, Hibiscus austroyunnanensis, Hibiscus barbosae, Hibiscus benguellensis, Hibiscus berberidifolius, Hibiscus bernieri, Hibiscus bifurcates, Hibiscus biseptus, Hibiscus bojerianus, Hibiscus boryanus, Hibiscus brackenridgei A. Gray, Hibiscus burtt-davyi, Hibiscus caerulescens, Hibiscus caesius, Hibiscus calyphyllus, Hibiscus cameronii, Hibiscus cannabinus L., Hibiscus castroi, Hibiscus cisplatinus, Hibiscus citrinus, Hibiscus clayi O. Deg. & I. Deg., Hibiscus clypeatus, Hibiscus coccineus (Medik.) Walter, Hibiscus colimensis, Hibiscus columnaris, Hibiscus comoensis, Hibiscus congestiflorus, Hibiscus costatus, Hibiscus coulteri, Hibiscus cuanzensis, Hibiscus dasycalyx, Hibiscus denudatus Benth., Hibiscus dimidiatus, Hibiscus dioscorides A.G. Mill., Hibiscus diplocrater, Hibiscus diriffan A.G. Mill., Hibiscus diversifolius, Hibiscus dongolensis, Hibiscus donianus, Hibiscus elatus, Hibiscus elegans, Hibiscus engleri, Hibiscus escobariae, Hibiscus excellii, Hibiscus ferrugineus, Hibiscus ficalhoanus, Hibiscus flavoroseus, Hibiscus fragilis DC., Hibiscus fragrans, Hibiscus fritzscheae, Hibiscus fugosioides, Hibiscus furcellatus Desr., Hibiscus fuscus, Hibiscus genevii Bojer, Hibiscus gilletii, Hibiscus gossweileri, Hibiscus grandidieri, Hibiscus grandiflorus Michx., Hibiscus grandistipulatus, Hibiscus grewiifolius, Hibiscus hamabo, Hibiscus hastatus, Hibiscus heterophyllus, Hibiscus hirtus, Hibiscus hispidissimus, Hibiscus huellensis, Hibiscus hybridus, Hibiscus indicus, Hibiscus insularis Endl., Hibiscus integrifolius, Hibiscus jaliscensis, Hibiscus kochii, Hibiscus kokio, Hibiscus labordei, Hibiscus laevis All., Hibiscus lasiocarpos, Hibiscus lasiococcus, Hibiscus lavaterioides, Hibiscus laxiflorus, Hibiscus leptocladus, Hibiscus leviserninus, Hibiscus lilacinus, Hibiscus liliiflorus, Hibiscus longifolius, Hibiscus longisepalus, Hibiscus ludwigii, Hibiscus lunariifolius, Hibiscus macilwraithensis, Hibiscus macrogonus, Hibiscus macrophyllus, Hibiscus macropodus, Hibiscus makinoi, Hibiscus malacophyllus Balf., Hibiscus malacospermus, Hibiscus martianus, Hibiscus moscheutos L., Hibiscus mutabilis L., Hibiscus paramutabilis, Hibiscus pedunculatus, Hibiscus pernambucensis, Hibiscus phoeniceus, Hibiscus platanifolius, Hibiscus quattenensis, Hibiscus poeppigii, Hibiscus radiates, Hibiscus rosa-sinensis L., Hibiscus sabdariffa L., Hibiscus schizopetalus, Hibiscus scottii, Hibiscus socotranus, Hibiscus sinosyriacus, Hibiscus splendens, Hibiscus stenanthus Balf., Hibiscus striatus, Hibiscus sturtii, Hibiscus syriacus L., Hibiscus taiwanensis S.Y. Hu, Hibiscus tiliaceus L., Hibiscus tozerensis, Hibiscus trilobus, Hibiscus trionum L., Hibiscus vitifolius, Hibiscus waimeae A. Heller, or a combination thereof. The Hibiscus seed oil can include or be Hibiscus sabdariffa seed oil, which can be a natural source of gamma-tocopherol (vitamin E), omega-6 fatty acids, and omega-3 fatty acids. The Hibiscus seed oil can be 0.0001 wt % to 5 wt % of the solid cosmetic, or 0.001 wt % to 1 wt %, or less than or equal to 5 wt % but greater than or equal to 0.0001 wt %, 0.0005, 0.001, 0.005, 0.01, 0.05, 0.1, 0.5, 1, 2, 3, or 4 wt %.

The phase B component can include a licorice extract or a derivative thereof. Licorice is the common name of Glycyrrhiza glabra, a flowering plant of the bean family Fabaceae, from the root of which a sweet, aromatic flavoring can be extracted. The licorice extract or derivative thereof can include glycyrrhetinic acid, glycyrrhizic acid, a derivative thereof, or a combination thereof. The licorice extract or derivative thereof can include or be stearyl glycyrrhetinate, which is a pentacyclic triterpenoid derivative of the beta-amyrin type obtained from the hydrolysis of glycyrrhizic acid, which was obtained from the herb licorice. It is used in flavoring and it masks the bitter taste of drugs like aloe and quinine. It is effective in the treatment of peptic ulcer and also has expectorant (antitussive) properties. It has some additional pharmacological properties with possible antiviral, antifungal, antiprotozoal, and antibacterial activities, and can have calming or soothing properties. The licorice extract or derivative thereof can be 0.001 wt % to 5 wt % of the solid cosmetic, or 0.01 wt % to 1 wt % of the solid cosmetic, or less than or equal to 5 wt % but greater than or equal to 0.001 wt %, 0.005, 0.01, 0.05, 0.1, 0.5, 1, 2, 3, or 4 wt %.

The phase B component can include a filler. The filler can be any suitable filler. The filler can include a light-diffusing filler that enhances the appearance of the solid cosmetic (e.g., enhances transparency/translucency). The filler can be or include silica, which is a naturally occurring mineral (SiO₂) found in the Earth's crust. The filler can be 0.001 wt % to 5 wt % of the solid cosmetic, or 0.01 wt % to 1 wt % of the solid cosmetic, or less than or equal to 5 wt % but greater than or equal to 0.001 wt %, 0.005, 0.01, 0.05, 0.1, 0.5, 1. 2, 3, or 4 wt %.

The phase B component can include a flowering tree bark extract. The flowering tree bark extract can include or be a Magnolia tree bark extract. Magnolia tree bark extract is taken from Magnolia officinalis (commonly called houpu Magnolia or Magnolia-bark), a species of Magnolia native to the mountains and valleys of China at altitudes of 300-1500 m. The highly aromatic bark is stripped from the stems, branches, and roots and used in traditional Chinese medicine. The traditional use indications are to eliminate damp and phlegm, and relieve distension. The bark contains magnolol and honokiol, two poly phenolic compounds that have been demonstrated as peroxisome proliferator-activated receptor gamma (PPAR gamma) agonists and GABAA modulators. Preclinical studies have evaluated their various potential applications including antioxidant, anti-inflammatory, antitumor, and antimicrobial properties. Magnolia bark extract can have free radial-scavenging properties. The flowering tree bark extract can be 0.0001 wt % to 5 wt % of the solid cosmetic, or 0.001 wt % to 1 wt %, or less than or equal to 5 wt % but greater than or equal to 0.0001 wt %, 0.0005, 0.001, 0.005, 0.01, 0.05, 0.1, 0.5, 1, 2, 3, or 4 wt %.

The solid cosmetic can include a phase C component. The phase C component can include one or more components. The phase C component can be 0.001 wt % to 5 wt % of the solid cosmetic, or 0.01 wt % to 1 wt %, or less than or equal to 5 wt % but equal to or greater than 0.001 wt %, 0.005, 0.01, 0.05, 0.1, 0.2, 0.3, 0.4, 0.5, 0.8, 1, 1.5, 2, 2.5, 3, or 4 wt©. The phase C component can include any one or more suitable plant-based components. The phase C component can include a tansy oil, an Annonaceae flower oil, an Oleaceae flower oil, a chamomile extract, or a combination thereof.

The phase C component can include a tansy oil. The tansy oil can include an oil of Tanacetum abrotanifolium (L.) Druce, Tanacetum abrotanoides, Tanacetum achilleifolium (M. Bieb.) Sch. Bip., Tanacetum alatavicum, Tanacetum annuum, Tanacetum argenteum (Lam.) Willd., Tanacetum atkinsonii, Tanacetum balsamita L., Tanacetum bipinnatum (L.) Sch. Bip., Tanacetum camphoratum Less., Tanacetum cinerariifolium (Trevir.) Sch. Bip., Tanacetum coccineum (Willd.) Grierson, Tanacetum corymbosum (L.) Sch. Bip., Tanacetum densum (Labill.) Sch. Bip., Tanacetum falconeri, Tanacetum ferulaceum (Sch. Bip.) Tanacetum haradjanii (Rech. f.) Grierson, Tanacetum kaschgarianum, Tanacetum krylovianum, Tanacetum macrophyllum (Waldst. & Kit.) Sch. Bip., Tanacetum microphyllum DC., Tanacetum niveum, Tanacetum parthenifolium (Willd.) Sch. Bip., Tanacetum parthenium (L.) Sch. Bip., Tanacetum pinnatum, Tanacetum polycephalum, Tanacetum poteriifolium (Nordm.) Grierson, Tanacetum praeteritium (Horw.) Heywood, Tanacetum ptarmiciflorum (Webb) Sch. Bip., Tanacetum pulchrum, Tanacetum richterioides, Tanacetum santolina, Tanacetum scopulorum, Tanacetum tanacetoides, Tanacetum tatsienense, Tanacetum vulgare L, or a combination thereof. The tansy oil can include or be an oil of Tanacetum annum (Tanacetum annum essential oil), also called blue tansy oil. Blue tansy oil is derived from Moroccan Tanacetum annuum flowers in the family of Asteraceae, and contains naturally high levels of chamazulene, mycerine, sabinene, and camphor, which can have calming properties. The tansy oil can be 0.0001 wt % to 5 wt % of the solid cosmetic, or 0.001 wt % to 1 wt %, or less than or equal to 5 wt % but greater than or equal to 0.0001 wt %, 0.0005, 0.001, 0.005, 0.01, 0.05, 0.1, 0.5, 1, 2, 3, or 4 wt %.

The phase C component can include an Annonaceae flower oil. The Annonaceae flower oil can include an oil of a flower of Anaxagoreoideae, Anaxagorea, Ambavioideae, Annonoideae, Bocageeae, Guatterieae, Duguetieae, Xylopieae, Annoneae, Monodoreae, Uvariaeae, Malmeoideae, Piptostigmateae, Maimeeae, Maasieae, Fenerivieae, Phoenicantheae, Dendrokingstonieae, Monocarpieae, Miliuseae, or a combination thereof. The Annonaceae flower oil can include or be Cananga Odorata Genuina flower extract, also called Ylang Ylang flower oil, which is oil derived from Cananga odorata flowers that imparts a unique scent. The Annonaceae flower oil can be 0.0001 wt % to 5 wt % of the solid cosmetic, or 0.001 wt % to 1 wt %, or less than or equal to 5 wt % but greater than or equal to 0.0001 wt %, 0.0005, 0.001, 0.005, 0.01, 0.05, 0.1, 0.5, 1, 2, 3, or 4 wt % of the solid cosmetic.

The phase C component can include an Oleaceae flower oil. The Oleaceae are a family of flowering plants in the order Lamiales. The Oleaceae flower oil can include oil of a flower of Myxopyreae, Forsythieae, Fontanesieae, Jasniineae, Oleeae, or a combination thereof. The Oleaceae flower oil can include or be Jasmine oil, such as Egyptian jasmine oil. Jasmine oil can impart a unique scent. The Oleaceae flower oil can be 0.0001 wt % to 5 wt % of the solid cosmetic, or 0.001 wt % to 1 wt %, or less than or equal to 5 wt % but greater than or equal to 0.0001 wt %, 0.0005, 0,001, 0.005, 0.01, 0.05, 0.1, 0.5, 1, 2, 3, or 4 wt % of the solid cosmetic.

The phase C component can include a chamomile extract. The chamomile extract can include an extract of Matricaria chamomilla, Chamaemelum nobile, Chamomilla recutita, Anthemis arvensis, Anthemis cotula, Cladanthus mixtus, Chatnaemelum nobile, Cota tinctoria, Eriocephalus punctulatus, Matricaria discoidea, Tripleurospermum inodorum, or a combination thereof. The chamomile extract can include or be Chamomilla recutita extract, also called blue chamomile oil, which contains naturally high levels of chamazulene and bisabolol, both of which can have calming properties. The Both are known as calming compounds. The chamomile extract can be 0.0001 wt % to 5 wt % of the solid cosmetic, or 0.001 wt % to 1 wt %, or less than or equal to 5 wt % but greater than or equal to 0.0001 wt %, 0.0005, 0.001, 0.005, 0.01, 0.05, 0.1, 0.5, 1, 2, 3, or 4 wt % of the solid cosmetic.

### Method of Making the Solid Cosmetic

Various aspects of the present invention provide a method of making an embodiment of the solid cosmetic described herein. The method of making the solid cosmetic can include combining the amino acid oil-gelling agent and one or more oils including the fatty acid ester component including a (C₅-C₁₅)alkyl ester of a (C₁₄-C₂₀)fatty acid to form a mixture. The method can include heating the mixture to a temperature within and not exceeding a first temperature range of 145-160° C. sufficiently to substantially dissolve the amino acid oil-gelling agent in the one or more oils. The method can include adding the phase B component including an antioxidant, a plant oil, an evergreen flower extract, a Hibiscus seed oil, a licorice extract or derivative thereof, a filler, a flowering tree bark extract, or a combination thereof to the mixture and heating the mixture within and not exceeding the first temperature range sufficiently to achieve a translucent mixture. The method can include transferring the translucent mixture to molds while the translucent mixture is within and not less than a second temperature range of 140-155° C., to form the solid cosmetic.

The first temperature range can be 145-160° C., 150-158° C., or 154-156° C., or less than or equal to 160° C. but greater than or equal to 145° C., 146, 147, 148, 149, 150, 151, 152, 153, 154, 155, 156, 157, 158, or 159° C. The second temperature range can be 140-155° C., 145-153° C., or 147-149° C., or less than or equal to 155° C. but greater than or equal to 140° C., 141, 142, 143, 144, 145, 146, 147, 148, 149, 150, 151, 152, 153, or 154° C.

The transferring of the translucent mixture to the molds while the translucent mixture is within and not less than a second temperature range can include transferring the translucent mixture to the molds without any intermediary steps of storage, cooling below the second temperature range, and reheating. The transferring of the translucent mixture to the molds while the translucent mixture is within and not less than a second temperature range can include transferring the translucent mixture to the molds before the translucent mixture cools below 145° C., or below 147° C., or before the mixture cools below 140° C., 141, 142, 143, 144, 145, 146, 147, 148, 149, 150, 151, 152, 153, 154. or 155° C.

The method can further include adding the phase C component including a tansy oil, an Annonaceae flower oil, an Oleaceae flower oil, a chamomile extract, or a combination thereof to the translucent mixture prior to the transferring to the molds, and heating the mixture to within and not exceeding the second temperature range while agitating the mixture until it is again translucent prior to the transferring. The method can further include heating the phase C component to within a third temperature range less than the second temperature range prior to combining the phase C component with the translucent mixture. The third temperature range can be 40-60° C., or 45-55° C., or less than or equal to 60° C. but greater than or equal to 40° C., 41, 42, 43, 44, 45, 46, 47, 48, 49, 50, 51, 52, 53, 54, 55, 56, 57, 58, or 59° C.

The transferring of the mixture to the molds can be completed within 0.01-20 minutes of the achieving of the translucent mixture after the addition of the phase C component, or within 0.01-10 minutes, or within 0.01-5 minutes, or less than or equal to 20 minutes but greater than or equal to 0.01 minutes, 0.05, 0.1, 0.2, 0.4, 0.6, 0.8, 1, 1.5, 2, 2.5. 3, 4, 5, 6, 7, 8, 9, 10, 12, 14, 16, or 18 minutes of the achieving of the translucent mixture after the addition of the phase C component.

### Packaged Product

Various aspects of the present invention provide a packaged product. The packaged product includes an embodiment of the solid cosmetic described herein. The packaged product also includes packaging that is resistant to the absorption of oils from the solid cosmetic. The packaging can be wrapped around the solid cosmetic for shipping and handling prior to use.

The packaging can be any suitable packaging that resists the absorption of oils from the solid cosmetic. The packaging can include paper that includes a coating that resists absorption of oils from the solid cosmetic into the paper. The coating on the paper can be substantially free of silicon, fluorocarbons, plastics, BPA, or a combination thereof The paper can be free of non-recycled materials (e.g., the paper can be a fully recycled paper). The coating can include a soy-based UV-cured coating that resists absorption of oils from the solid cosmetic into the paper.

### Refillable Skin Care System

Various aspects of the present invention provide a refillable skin care system. The refillable skin care system includes an embodiment of the solid cosmetic described herein. The refillable skin care system also includes a refillable container for storing the solid cosmetic. The refillable container can be made of any suitable material. The refillable container can include a metal, a metal alloy, a plastic, or a combination thereof. The refillable container can include steel (e.g., any type of steel, such as stainless steel). The refillable containing can include a tin-coated steel (e.g., any type of tin-coated steel, such as tin-coated stainless steel).

### EXAMPLES

Various embodiments of the present invention can be better understood by reference to the following Examples which are offered by way of illustration. The present invention is not limited to the Examples given herein.

### Example 1. Production of Skin Treatment Bar

Table 1 illustrates the components used to form the skin treatment bar. All percentages in Table 1 are weight percentages unless otherwise indicated. “Final” percentages indicate wt % in the combined mixture of Phases A. B, and C. The ethylhexyl olivate was predominantly hexadecanoic acid (C16:0) and octadecenoic acid (C18:1 and C18:2) derived from olive oil fatty acids and esterified with ethylhexanol, and also included a small concentration of squalene.

TABLE 1
Components used to form skin treatment bar.

| | | Wt | Mass |
|---|---|---|---|
| Phase | Description | % | (g) |
| A | Simmondsia chinensis (Jojoba) seed oil | 40.97 | 348.245 |
| A | Ethylhexyl olivate (95%, final 39%) and | 40.90 | 347.65 |
| | squalene (5% or less, final 2.03% or less) | | |
| A | Dibutyl ethylhexanoyl glutamide | 9.51 | 80.835 |
| A | Dibutyl lauroyl glutamide | | |
| B | Tetrahexyldecyl ascorbate | 8.43 | 71.655 |
| B | Cocos Nucifera (coconut) oil (99.8%) and | | |
| | gardenia Tahitensis flowers extract (0.2%, final | | |
| | 0.01%) | | |
| B | Hibiscus Sabdariffa (Hibiscus) seed oil | | |
| B | Stearyl glycyrrhetinate | | |
| B | Silica | | |
| B | Magnolia Officinalis bark extract | | |
| C | Tanacetum annum (Tansy) essential oil | 0.19 | 1.615 |
| C | Cananga Odorata Genuina flower oil | | |
| C | Jasminum Officinale oil | | |
| C | Chamomilla Recutita | | |

| | | |
|---|---|---|
| TOTAL | 100.000 | 850 |

The Jojoba oil and ethylhexyl olivate were weighed into a main compounding vessel. The composition was continuously mixed using a magnetic stirring bar, and was heated to 148° C. The dibutyl ethylhexanoyl glutamide and dibutyl lauroyl glutamide were weighed out in a separate vessel and were slowly sifted into the main compounding vessel with vigorous mixing until no powder was visible on top of the composition, The main compounding vessel was heated to 150-155° C. (155° C. was not exceeded). Preparation of Phase A materials was complete once substantially all the glutamide particles were dissolved on inspection with the naked eye.

The Phase B components were weighted together in a separate vessel. After combining all the Phase B components, mixing at 50° C. was initiated and was continued until the mixture liquified and was translucent and nearly clear. The Phase B mixture was poured into the main compounding vessel, causing the temperature of the composition in the main compounding vessel to slightly drop. The main compounding vessel was stirred and was heated to 150-155° C. without exceeding 155° C. until the composition therein appeared clear and uniform. Once this occurred, the heating equipment was turned off, and the mixing was continued.

The Phase C components were weighed and combined together. The Phase C mixture was added to the Phase A/Phase B mixture in the main compounding vessel that has the heat turned off. Phase A, B, and C were mixed together in the main compounding vessel until the mixture was clear and uniform.

The mixture was then directly poured into silicone molds while the mixture was at 148-150° C. The pouring procedure was completed within 5 minutes. The product was allowed to cool for a minimum of 3 hours, and was then removed from the molds as solid bars. The bars were then processed for packaging.

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present invention. Thus, it should be understood that although the present invention has been specifically disclosed by specific embodiments and optional features, modification and variation of the concepts herein disclosed may be resorted to by those of ordinary skill in the art, and that such modifications and variations are considered to be within the scope of embodiments of the present invention.

### Exemplary Embodiments

The following exemplary embodiments are provided, the numbering of which is not to be construed as designating levels of importance:

Embodiment 1 provides a solid cosmetic comprising:
an amino acid oil-gelling agent; and
a fatty acid ester component comprising a (C₅-C₁₅)alkyl ester of a (C₁₄-C₂₀)fatty acid;
wherein the solid cosmetic is substantially free of water and polyamides.

Embodiment 2 provides the solid cosmetic of Embodiment 1, wherein the solid cosmetic is transparent or translucent.

Embodiment 3 provides the solid cosmetic of any one of Embodiments 1-2, wherein the solid cosmetic is free of suspended particles by inspection with the naked eye.

Embodiment 4 provides the solid cosmetic of any one of Embodiments 1-3, wherein the solid cosmetic is free of synthetic dyes.

Embodiment 5 provides the solid cosmetic of any one of Embodiments 1-4, wherein less than 0.01 wt % of the solid cosmetic is synthetic dyes, or less than 0.001 wt %, or less than 0.0001 wt %.

Embodiment 6 provides the solid cosmetic of any one of Embodiments 1-5, wherein the solid cosmetic is free of synthetic fragrances.

Embodiment 7 provides the solid cosmetic of any one of Embodiments 1-6, wherein less than 0.01 wt % of the solid cosmetic is synthetic fragrances, or less than 0.001 wt %, or less than 0.0001 wt %.

Embodiment 8 provides the solid cosmetic of any one of Embodiments 1-7, wherein the solid cosmetic is free of water.

Embodiment 9 provides the solid cosmetic of any one of Embodiments 1-8, wherein less than 0.01 wt % of the solid cosmetic is water, or less than 0.001 wt %, or less than 0.0001 wt %.

Embodiment 10 provides the solid cosmetic of any one of Embodiments 1-9, wherein the solid cosmetic is free of polyamides.

Embodiment 11 provides the solid cosmetic of any one of Embodiments 1-10, wherein less than 0.01 wt % of the solid cosmetic is polyamides, or less than 0.001 wt %, or less than 0.0001 wt %.

Embodiment 12 provides the solid cosmetic of any one of Embodiments 1-11, wherein the (C₅-C₁₅)alkyl of the (C₁₄-C₂₀)fatty acid is branched or unbranched.

Embodiment 13 provides the solid cosmetic of any one of Embodiments 1-12, wherein the (C₅-C₁₅)alkyl of the (C₁₄-C₂₀)fatty acid is branched.

Embodiment 14 provides the solid cosmetic of any one of Embodiments 1-13, wherein the (C₅-C₁₅)alkyl ester of the (C₁₄-C₂₀)fatty acid is a (C₇-C₁₀)alkyl ester.

Embodiment 15 provides the solid cosmetic of any one of Embodiments 1-14, wherein the (C₅-C₁₅)alkyl ester of the (C₁₄-C₂₀)fatty acid is a (C₈)alkyl ester.

Embodiment 16 provides the solid cosmetic of any one of Embodiments 1-15, wherein the (C₅-C₁₅)alkyl ester of the (C₁₄-C₂₀)fatty acid is ethylhexyl.

Embodiment 17 provides the solid cosmetic of any one of Embodiments 1-16, wherein the (C₁₄-C₂₀)fatty acid is a (C₁₆-C₁₈)fatty acid.

Embodiment 18 provides the solid cosmetic of any one of Embodiments 1-17, wherein the (C₁₄-C₂₀)fatty acid is a (C₁₆)fatty acid.

Embodiment 19 provides the solid cosmetic of any one of Embodiments 1-18, wherein the (C₁₄-C₂₀)fatty acid is a (C₁₈) fatty acid.

Embodiment 20 provides the solid cosmetic of any one of Embodiments 1-19, wherein the fatty acid ester component comprises a (C₅-C₁₅)alkyl ester of a (C₁₆)fatty acid and a (C₅-C₁₅)alkyl ester of a (C₁₈)fatty acid.

Embodiment 21 provides the solid cosmetic of any one of Embodiments 1-20, wherein the fatty acid ester component comprises ethylhexyl hexadecanoic acid, ethylhexyl palmitate, ethylhexyl linoleate, or a combination thereof.

Embodiment 22 provides the solid cosmetic of any one of Embodiments 1-21, wherein the fatty acid ester component is 20 wt % to 60 wt % of the solid cosmetic.

Embodiment 23 provides the solid cosmetic of any one of Embodiments 1-22, wherein the fatty acid ester component is 35 wt % to 45 wt % of the solid cosmetic.

Embodiment 24 provides the solid cosmetic of any one of Embodiments 1-23, wherein the fatty acid ester component comprises ethylhexyl olivate.

Embodiment 25 provides the solid cosmetic of Embodiment 24, wherein the ethylhexyl olivate is 20 wt % to 60 wt % of the solid cosmetic.

Embodiment 26 provides the solid cosmetic of any one of Embodiments 24-25, wherein the ethylhexyl olivate is 35 wt % to 45 wt % of the solid cosmetic.

Embodiment 27 provides the solid cosmetic of any one of Embodiments 1-26, wherein the amino acid oil-gelling agent is 1 wt % to 30 wt % of the solid cosmetic.

Embodiment 28 provides the solid cosmetic of any one of Embodiments 1-27, wherein the amino acid oil-gelling agent is 5 wt % to 15 wt % of the solid cosmetic.

Embodiment 29 provides the solid cosmetic of any one of Embodiments 1-28, wherein the amino acid oil-gelling agent comprises more than one amino acid oil-gelling agent.

Embodiment 30 provides the solid cosmetic of any one of Embodiments 1-29, wherein the amino acid oil-gelling agent comprises dibutyl lauroyl glutamide.

Embodiment 31 provides the solid cosmetic of Embodiment 30, wherein the dibutyl lauroyl glutamide is 1 wt % to 30 wt % of the solid cosmetic.

Embodiment 32 provides the solid cosmetic of any one of Embodiments 30-31, wherein the dibutyl lauroyl glutamide is 2 wt % to 12 wt % of the solid cosmetic.

Embodiment 33 provides the solid cosmetic of any one of Embodiments 1-31, wherein the amino acid oil-gelling agent comprises dibutyl ethylhexanoyl glutamide.

Embodiment 34 provides the solid cosmetic of Embodiment 33, wherein the dibutyl ethylhexanoyl glutamide is 0.1 wt % to 10 wt % of the solid cosmetic.

Embodiment 35 provides the solid cosmetic of any one of Embodiments 33-34, wherein the dibutyl ethylhexanoyl glutamide is 1 wt % to 5 wt % of the solid cosmetic.

Embodiment 36 provides the solid cosmetic of any one of Embodiments 1-35, wherein the amino acid oil-gelling agent comprises dibutyl lauroyl glutamide and dibutyl ethylhexanoyl glutamide.

Embodiment 37 provides the solid cosmetic of Embodiment 36, wherein the dibutyl lauroyl glutamide and dibutyl ethylhexanoyl glutamide are about 1 wt % to 30 wt % of the solid cosmetic.

Embodiment 38 provides the solid cosmetic of any one of Embodiments 36-37, wherein the dibutyl lauroyl glutamide and dibutyl ethylhexanoyl glutamide are about 5 wt % to 15 wt % of the solid cosmetic.

Embodiment 39 provides the solid cosmetic of any one of Embodiments 36-38, wherein a weight ratio of the dibutyl lauroyl glutamide to the dibutyl ethylhexanoyl glutamide is 1:10 to 10:1.

Embodiment 40 provides the solid cosmetic of any one of Embodiments 36-39, wherein a weight ratio of the dibutyl lauroyl glutamide to the dibutyl ethylhexanoyl glutamide is 1:1 to 5:1.

Embodiment 41 provides the solid cosmetic of any one of Embodiments 1-40, further comprising one or more seed or vegetable oils.

Embodiment 42 provides the solid cosmetic of Embodiment 41, wherein the one or more seed or vegetable oils, the amino acid oil-gelling agent, and the fatty acid ester component, are together 70 wt % to 99 wt % of the solid cosmetic.

Embodiment 43 provides the solid cosmetic of any one of Embodiments 41-42, wherein the one or more seed or vegetable oils, the amino acid oil-gelling agent, and the fatty acid ester component, are together 85 wt % to 95 wt % of the solid cosmetic.

Embodiment 44 provides the solid cosmetic of any one of Embodiments 41-43, wherein the one or more seed or vegetable oils are 20 wt % to 60 wt % of the solid cosmetic.

Embodiment 45 provides the solid cosmetic of any one of Embodiments 41-44, wherein the one or more seed or vegetable oils are 30 wt % to 50 wt % of the solid cosmetic.

Embodiment 46 provides the solid cosmetic of any one of Embodiments 41-45, wherein the one or more seed or vegetable oils comprise coconut oil, corn oil, canola oil, cottonseed oil, olive oil, palm oil, peanut oil, rapeseed oil, safflower oil, sesame oil, soybean oil, sunflower oil, cocoa butter, shea butter, mango butter, Jojoba oil, a fraction thereof, a middle or high oleic version thereof, a hydrogenated oil formed therefrom, an esterified or interesterified oil formed therefrom, or a combination thereof.

Embodiment 47 provides the solid cosmetic of any one of Embodiments 41-46, wherein the one or more seed or vegetable oils comprise Jojoba oil.

Embodiment 48 provides the solid cosmetic of Embodiment 47, wherein the Jojoba oil is 20 wt % to 60 wt % of the solid cosmetic.

Embodiment 49 provides the solid cosmetic of any one of Embodiments 47-48, wherein the Jojoba oil is 30 wt % to 50 wt % of the solid cosmetic.

Embodiment 50 provides the solid cosmetic of any one of Embodiments 1-49, wherein the solid cosmetic comprises a triterpene.

Embodiment 51 provides the solid cosmetic of Embodiment 50, wherein the triterpene comprises squalene, and/or comprises a ring skeleton corresponding to polypodatetraene, malabaricane, lanostane, cucurbitacin, hopane, oleanane, ursolic acid, or a combination thereof.

Embodiment 52 provides the solid cosmetic of any one of Embodiments 50-51, wherein the triterpene comprises squalene.

Embodiment 53 provides the solid cosmetic of any one of Embodiments 50-52, wherein the triterpene is 0.1 wt % to 10 wt % of the solid cosmetic.

Embodiment 54 provides the solid cosmetic of any one of Embodiments 50-53, wherein the triterpene is 1 wt % to 5 wt % of the solid cosmetic.

Embodiment 55 provides the solid cosmetic of any one of Embodiments 1-54, further comprising a phase B component.

Embodiment 56 provides the solid cosmetic of Embodiment 55, wherein the phase B component is 1 wt % to 30 wt % of the solid cosmetic.

Embodiment 57 provides the solid cosmetic of any one of Embodiments 55-56, wherein the phase B component is 5 wt % to 15 wt % of the solid cosmetic.

Embodiment 58 provides the solid cosmetic of any one of Embodiments 55-57, wherein the phase B component comprises one or more antioxidants.

Embodiment 59 provides the solid cosmetic of Embodiment 58, wherein the one or more antioxidants comprises ascorbic acid, glutathione, lipoid acid, uric acid, a carotene, alpha tocopherol, ubiquinol, a derivative thereof, or a combination thereof.

Embodiment 60 provides the solid cosmetic of any one of Embodiments 58-59, wherein the one or more antioxidants comprises tetrahexyldecyl ascorbate.

Embodiment 61 provides the solid cosmetic of any one of Embodiments 58-60, wherein the one or more antioxidants are 0.01 wt % to 10 wt % of the solid cosmetic.

Embodiment 62 provides the solid cosmetic of any one of Embodiments 58-61, wherein the one or more antioxidants are 0.1 wt % to 5 wt % of the solid cosmetic.

Embodiment 63 provides the solid cosmetic of any one of Embodiments 55-62, wherein the phase B component comprises one or more plant oils.

Embodiment 64 provides the solid cosmetic of Embodiment 63, wherein the one or more plant oils comprises coconut oil, corn oil, canola oil, cottonseed oil, olive oil, palm oil, peanut oil, rapeseed oil, safflower oil, sesame oil, soybean oil, sunflower oil, cocoa butter, shea butter, mango butter, Jojoba oil, a fraction thereof, a middle or high oleic version thereof, a hydrogenated oil formed therefrom, an esterified or interesterified oil formed therefrom, or a combination thereof.

Embodiment 65 provides the solid cosmetic of any one of Embodiments 63-64, wherein the one or more plant oils comprises Cocos Nucifera oil.

Embodiment 66 provides the solid cosmetic of any one of Embodiments 63-65, wherein the one or more plant oils are 1 wt % to 20 wt % of the solid cosmetic.

Embodiment 67 provides the solid cosmetic of any one of Embodiments 63-66, wherein the one or more plant oils are 5 wt % to 10 wt % of the solid cosmetic.

Embodiment 68 provides the solid cosmetic of any one of Embodiments 55-67, wherein the phase B component comprises one or more evergreen flower extracts.

Embodiment 69 provides the solid cosmetic of Embodiment 68, wherein the one or more evergreen flower extracts comprises Gardenia Tahitensis flowers extract.

Embodiment 70 provides the solid cosmetic of any one of Embodiments 68-69, wherein the one or more evergreen flower extracts are 0.0001 wt % to 5 wt % of the solid cosmetic.

Embodiment 71 provides the solid cosmetic of any one of Embodiments 68-70, wherein the one or more evergreen flower extracts are 0.001 wt % to 1 wt % of the solid cosmetic.

Embodiment 72 provides the solid cosmetic of any one of Embodiments 55-71, wherein the phase B component comprises a Hibiscus seed oil.

Embodiment 73 provides the solid cosmetic of Embodiment 72, wherein the Hibiscus seed oil comprises an oil of Hibiscus abelmoschus L., Hibiscus abelmoschus var. betulifolius Mast., Hibiscus abelmoschus var. genuinus Hochr., Hibiscus laevis abutiloides Willd., Hibiscus abyssinicus Steud., Hibiscus acapulcensis Fryxell, Hibiscus acerifolius Salisb., Hibiscus acerifolius DC., Hibiscus acetosaefolius DC., Hibiscus acetosella Welw. ex Hiern., Hibiscus acicularis, Hibiscus aculeatus, Hibiscus latissimus, Hibiscus andongensis, Hibiscus angolensis, Hibiscus aponeurus, Hibiscus archeri, Hibiscus aridicola, Hibiscus arnottianus A. Gray, Hibiscus asper, Hibiscus austroyunnanensis, Hibiscus barbosae, Hibiscus benguellensis, Hibiscus berberidifolius, Hibiscus bernieri, Hibiscus bifurcates, Hibiscus biseptus, Hibiscus bojerianus, Hibiscus boryanus, Hibiscus brackenridgei A. Gray, Hibiscus burtt-davyi, Hibiscus caerulescens, Hibiscus caesius, Hibiscus calyphyllus, Hibiscus cameronii, Hibiscus cannabinus L., Hibiscus castroi, Hibiscus cisplatinus, Hibiscus citrinus, Hibiscus clayi O. Deg. & I. Deg., Hibiscus clypeatus, Hibiscus coccineus (Medik.) Walter, Hibiscus colitnensis, Hibiscus columnaris, Hibiscus comoensis, Hibiscus congestiflorus, Hibiscus costatus, Hibiscus coulteri, Hibiscus cuanzensis, Hibiscus dasycalyx, Hibiscus denudates Benth., Hibiscus dimidiatus, Hibiscus dioscorides A.G. Mill., Hibiscus diplocrater, Hibiscus diriffan A.G. Mill., Hibiscus diversifolius, Hibiscus dongolensis, Hibiscus donianus, Hibiscus elatus, Hibiscus elegans, Hibiscus engleri, Hibiscus escobariae, Hibiscus excellii, Hibiscus ferrugineus, Hibiscus ficalhoanus, Hibiscus flavoroseus, Hibiscus fragilis DC., Hibiscus fragrans, Hibiscus fritzscheae, Hibiscus fugosioides, Hibiscus furcellatus Desr., Hibiscus fuscus, Hibiscus genevii Bojer, Hibiscus gilletii, Hibiscus gossweileri, Hibiscus grandidieri, Hibiscus grandiflorus Michx., Hibiscus grandistipulatus, Hibiscus grewiifolius, Hibiscus hamabo, Hibiscus hastatus, Hibiscus heterophyllus, Hibiscus hirtus, Hibiscus hispidissimus, Hibiscus huellensis, Hibiscus hybridus, Hibiscus indicus, Hibiscus insularis Endi., Hibiscus integrifolius, Hibiscus jaliscensis, Hibiscus kochii, Hibiscus kokio, Hibiscus labordei, Hibiscus laevis All., Hibiscus lasiocarpos, Hibiscus lasiococcus, Hibiscus lavaterioides, Hibiscus laxiflorus, Hibiscus leptociadus, Hibiscus leviserninus, Hibiscus lilacinus, Hibiscus liliiflorus, Hibiscus longifolius, Hibiscus longisepalus, Hibiscus ludwigii, Hibiscus lunariifolius, Hibiscus macilwraithensis, Hibiscus macrogonus, Hibiscus macrophyllus, Hibiscus macropodus, Hibiscus makinoi, Hibiscus malacophyllus Balf., Hibiscus malacospermus, Hibiscus martianus, Hibiscus moscheutos L., Hibiscus mutabilis L., Hibiscus paramutabilis, Hibiscus pedunculatus, Hibiscus pernambucensis, Hibiscus phoeniceus, Hibiscus platanifolius, Hibiscus quattenensis, Hibiscus poeppigii, Hibiscus radiates, Hibiscus rosa-sinensis L., Hibiscus sabdariffa. L., Hibiscus schizopetalus, Hibiscus scottii, Hibiscus socotranus, Hibiscus sinosyriacus, Hibiscus splendens, Hibiscus stenanthus Balf., Hibiscus striates, Hibiscus sturtii, Hibiscus syriacus L., Hibiscus taiwanensis S. Y. Hu, Hibiscus tiliaceus L., Hibiscus tozerensis, Hibiscus trilobus, Hibiscus trionurn L., Hibiscus vitifolius, Hibiscus waimeae A. Heller, or a combination thereof.

Embodiment 74 provides the solid cosmetic of any one of Embodiments 72-73, wherein the Hibiscus seed oil comprises Hibiscus sabdarifia seed oil.

Embodiment 75 provides the solid cosmetic of any one of Embodiments 72-74, wherein the Hibiscus seed oil is 0.0001 wt % to 5 wt % of the solid cosmetic.

Embodiment 76 provides the solid cosmetic of any one of Embodiments 72-75, wherein the Hibiscus seed oil is 0.001 wt % to 1 wt % of the solid cosmetic.

Embodiment 77 provides the solid cosmetic of any one of Embodiments 55-76, wherein the phase B component comprises a licorice extract or derivative thereof.

Embodiment 78 provides the solid cosmetic of Embodiment 77, wherein the licorice extract or derivative thereof comprises glycyrrhetinic acid, glycyrrhizic acid, a derivative thereof, or a combination thereof.

Embodiment 79 provides the solid cosmetic of any one of Embodiments 77-78, wherein the licorice extract or derivative thereof comprises stearyl glycyrrhetinate.

Embodiment 80 provides the solid cosmetic of any one of Embodiments 77-79, wherein the licorice extract or derivative thereof is 0.001 wt % to 5 wt % of the solid cosmetic.

Embodiment 81 provides the solid cosmetic of any one of Embodiments 77-80, wherein the licorice extract or derivative thereof is 0.01 wt % to 1 wt % of the solid cosmetic.

Embodiment 82 provides the solid cosmetic of any one of Embodiments 55-81, wherein the phase B component comprises a filler.

Embodiment 83 provides the solid cosmetic of Embodiment 82, wherein the filler comprises a light-diffusing filler.

Embodiment 84 provides the solid cosmetic of any one of Embodiments 82-83, wherein the filler comprises silica.

Embodiment 85 provides the solid cosmetic of any one of Embodiments 82-84, wherein the filler is 0.001 wt % to 5 wt % of the solid cosmetic.

Embodiment 86 provides the solid cosmetic of any one of Embodiments 82-85, wherein the filler is 0.01 wt % to 1 wt % of the solid cosmetic.

Embodiment 87 provides the solid cosmetic of any one of Embodiments 55-96, wherein the phase B component comprises a flowering tree bark extract.

Embodiment 88 provides the solid cosmetic of Embodiment 87, wherein the flowering tree bark extract comprises a Magnolia tree bark extract.

Embodiment 89 provides the solid cosmetic of any one of Embodiments 87-88, wherein the flowering tree bark extract comprises Magnolia officinalis bark extract.

Embodiment 90 provides the solid cosmetic of any one of Embodiments 87-89, wherein the flowering tree bark extract is 0.0001 wt % to 5 wt % of the solid cosmetic.

Embodiment 91 provides the solid cosmetic of any one of Embodiments 87-90, wherein the flowering tree bark extract is 0.001 wt % to 1 wt % of the solid cosmetic.

Embodiment 92 provides the solid cosmetic of any one of Embodiments 1-91, further comprising a phase C component.

Embodiment 93 provides the solid cosmetic of Embodiment 92, wherein the phase C component is 0.001 wt % to 5 wt % of the solid cosmetic.

Embodiment 94 provides the solid cosmetic of any one of Embodiments 92-93, wherein the phase C component is 0.01 wt % to 1 wt % of the solid cosmetic.

Embodiment 95 provides the solid cosmetic of any one of Embodiments 92-94, wherein the phase C component comprises a tansy oil.

Embodiment 96 provides the solid cosmetic of Embodiment 95, wherein the tansy oil comprises an oil of Tanacetum abrotanifolium (L.) Druce, Tanacetum abrotanoides, Tanacetum achilleifolium (M. Bieb.) Sch. Bip., Tanacetum alatavicum, Tanacetum annuum, Tanacetum argenteum (Lam.) Willd., Tanacetum atkinsonii, Tanacetum balsamita L., Tanacetum bipinnatum (L.) Sch. Bip., Tanacetum camphoratum Less., Tanacetum cinerariifolium (Trevir.) Sch. Bip., Tanacetum coccineum (Willd.) Grierson, Tanacetum corymbosum (L.) Sch. Bip., Tanacetum densum (Labill.) Sch. Bip., Tanacetum falconeri, Tanacetum ferulaceum (Sch. Bip.) Walp., Tanacetum haradjanii (Rech. f.) Grierson, Tanacetum kaschgarianum, Tanacetum krylovianum, Tanacetum macrophyllum (Waldst. & Kit.) Sch. Bip. Tanacetum microphyllum DC., Tanacetum niveum, Tanacetum parthenifolium (Willd,) Sch. Bip., Tanacetum parthenium (L.) Sch. Bip., Tanacetum pinnatum, Tanacetum polycephalum, Tanacetum poteriifolium (Nordin.) Grierson, Tanacetum praeteritium (Horw.) Heywood, Tanacetum ptarmiciflorum (Webb) Sch. Bip., Tanacetum pulchrum, Tanacetum richterioides, Tanacetum santolina, Tanacetum scopulorum, Tanacetum tanacetoides, Tanacetum tatsienense, Tanacetum vulgare L., or a combination thereof.

Embodiment 97 provides the solid cosmetic of any one of Embodiments 95-96, wherein the tansy oil comprises Tanacetum annum essential oil.

Embodiment 98 provides the solid cosmetic of any one of Embodiments 95-97, wherein the tansy oil is 0.0001 wt % to 5 wt % of the solid cosmetic.

Embodiment 99 provides the solid cosmetic of any one of Embodiments 95-98, wherein the tansy oil is 0.001 wt % to 1 wt % of the solid cosmetic.

Embodiment 100 provides the solid cosmetic of any one of Embodiments 92-99, wherein the phase C component comprises an Annonaceae flower oil.

Embodiment 101 provides the solid cosmetic of Embodiment 100, wherein the Annonaceae flower oil comprises an oil of a flower of Anaxagoreoideae, Anaxagorea, Ambavioideae, Annonoideae, Bocageeae, Guatterieae, Duguetieae, Xylopieae, Annoneae, Monodoreae, Uvariaeae, Malmeoideae, Piptostigmateae, Malmeeae, Maasieae, Fenerivieae, Phoenicantheae, Dendrokingstonieae, Monocarpieae, Miliuseae, or a combination thereof.

Embodiment 102 provides the solid cosmetic of any one of Embodiments 100-101, wherein the Annonaceae flower oil comprises Cananga Odorata Genuina flower extract.

Embodiment 103 provides the solid cosmetic of any one of Embodiments 100-102, wherein the Annonaceae flower oil is 0.0001 wt % to 5 wt % of the solid cosmetic.

Embodiment 104 provides the solid cosmetic of any one of Embodiments 100-103, wherein the Annonaceae flower oil is 0.001 wt % to 1 wt % of the solid cosmetic.

Embodiment 105 provides the solid cosmetic of any one of Embodiments 92-104, wherein the phase C component comprises an Oleaceae flower oil.

Embodiment 106 provides the solid cosmetic of Embodiment 105, wherein the Oleaceae flower oil comprises oil of a flower of Myxopyreae, Forsythieae, Fontanesieae, Jasmineae, Oleeae, or a combination thereof.

Embodiment 107 provides the solid cosmetic of any one of Embodiments 105-106, wherein the Oleaceae flower oil comprises Jasmine oil.

Embodiment 108 provides the solid cosmetic of any one of Embodiments 105-107, wherein the Oleaceae flower oil is 0.0001 wt % to 5 wt % of the solid cosmetic.

Embodiment 109 provides the solid cosmetic of any one of Embodiments 105-108, wherein the Oleaceae flower oil is 0.001 wt % to 1 wt % of the solid cosmetic.

Embodiment 110 provides the solid cosmetic of any one of Embodiments 92-109, wherein the phase C component comprises a chamomile extract.

Embodiment 111 provides the solid cosmetic of Embodiment 110, wherein the chamomile extract comprises an extract of Matricaria chamomilla, Chamaemelum nobile, Chamomilla recutita, Anthemis arvensis, Anthemis cotula, Cladanthus mixtus, Chamaemelum nobile, Cota tinctoria, Eriocephalus punctulatus, Matricaria discoidea, Tripleurospermum inodorum, or a combination thereof.

Embodiment 112 provides the solid cosmetic of any one of Embodiments 110-111, wherein the chamomile extract comprises Chamomilla recutita extract.

Embodiment 113 provides the solid cosmetic of any one of Embodiments 110-112, wherein the chamomile extract comprises chamomile blue oil.

Embodiment 114 provides the solid cosmetic of any one of Embodiments 110-113, wherein the chamomile extract is 0.0001 wt % to 5 wt % of the solid cosmetic.

Embodiment 115 provides the solid cosmetic of any one of Embodiments 110-114, wherein the chamomile extract is 0.001 wt % to 1 wt % of the solid cosmetic.

Embodiment 116 provides a solid cosmetic comprising:
an amino acid oil-gelling agent comprising dibutyl lauroyl glutamide, dibutyl ethylhexanoyl glutamide, or a combination thereof, wherein the amino acid oil-gelling agent is 1 wt % to 30 wt % of the solid cosmetic;
a fatty acid ester component comprising a (C₅-C₁₅)alkyl ester of a (C₁₆)fatty acid and a (C₅-C₁₅)alkyl ester of a (C₁₈)fatty acid, wherein the fatty acid ester component is 20 wt % to 60 wt % of the solid cosmetic; and
one or more seed or vegetable oils that are 20 wt % to 60 wt % of the solid cosmetic;
wherein water and polyamides are less than 0.01 wt % of the solid cosmetic.

Embodiment 117 provides a solid cosmetic comprising:
an amino acid oil-gelling agent comprising dibutyl lauroyl glutamide and dibutyl ethylhexanoyl glutamide having a weight ratio of 1:1 to 5:1, wherein the amino acid oil-gelling agent is 5 wt % to 15 wt % of the solid cosmetic;
a fatty acid ester component comprising ethylhexyl olivate, wherein the fatty acid ester component is 35 wt % to 45 wt % of the solid cosmetic; and
one or more seed or vegetable oils comprising Jojoba oil, wherein the jojoba, oil is 30 wt % to 50 wt % of the solid cosmetic;
wherein water and polyamides are less than 0.01 wt % of the solid cosmetic.

Embodiment 118 provides a solid cosmetic comprising:
an amino acid oil-gelling agent comprising dibutyl lauroyl glutamide, dibutyl ethylhexanoyl glutamide, or a combination thereof, wherein the amino acid oil-gelling agent is 1 wt % to 30 wt % of the solid cosmetic;
a fatty acid ester component comprising a (C₅-C₁₅)alkyl ester of a (C₁₆)fatty acid and a (C₅-C₁₅)alkyl ester of a (C₁₈)fatty acid, wherein the fatty acid ester component is 20 wt % to 60 wt % of the solid cosmetic;
one or more seed or vegetable oils that are 20 wt % to 60 wt % of the solid cosmetic;
a phase B component comprising an antioxidant, a plant oil, an evergreen flower extract, a Hibiscus seed oil, a licorice extract or derivative thereof, a filler, a flowering tree bark extract, or a combination thereof, wherein the phase B component is 1 wt % to 30 wt % of the solid cosmetic; and
a phase C component comprising a tansy oil, an Annonaceae flower oil, an Oleaceae flower oil, a chamomile extract, or a combination thereof, wherein the phase C component is 0.001 wt % to 5 wt % of the solid cosmetic;
wherein water and polyamides are less than 0.01 wt % of the solid cosmetic.

Embodiment 119 provides a solid cosmetic comprising:
an amino acid oil-gelling agent comprising dibutyl lauroyl glutamide, dibutyl ethylhexanoyl glutamide, or a combination thereof, wherein the amino acid oil-gelling agent is 1 wt % to 30 wt % of the solid cosmetic;
a fatty acid ester component comprising a (C₅-C₁₅)alkyl ester of a (C₁₆)fatty acid and a (C₅-C₁₅)alkyl ester of a (C₁₈)fatty acid, wherein the fatty acid ester component is 20 wt % to 60 wt % of the solid cosmetic;
one or more seed or vegetable oils that are 20 wt % to 60 wt % of the solid cosmetic;
a phase B component comprising an antioxidant, a plant oil, an evergreen flower extract, a Hibiscus seed oil, a licorice extract or derivative thereof, a filler, and a flowering tree bark extract, wherein the phase B component is 1 wt % to 30 wt % of the solid cosmetic; and
a phase C component comprising a tansy oil, an Annonaceae flower oil, an Oleaceae flower oil, and a chamomile extract, wherein the phase C component is 0.001 wt % to 5 wt % of the solid cosmetic;
wherein water and polyamides are less than 0.01 wt % of the solid cosmetic.

Embodiment 120 provides a solid cosmetic comprising:
an amino acid oil-gelling agent comprising dibutyl lauroyl glutamide and dibutyl ethylhexanoyl glutamide having a weight ratio of 1:1 to 5:1, wherein the amino acid oil-gelling agent is 5 wt % to 15 wt % of the solid cosmetic;
a fatty acid ester component comprising ethylhexyl olivate, wherein the fatty acid ester component is 35 wt % to 45 wt % of the solid cosmetic;
one or more seed or vegetable oils comprising Jojoba oil, wherein the Jojoba oil is 30 wt % to 50 wt % of the solid cosmetic;
a phase B component comprising tetrahexyldecyl ascorbate, Cocos Nucifera oil, Gardenia Tahitensis flowers extract, Hibiscus sabdariffa seed oil, stearyl glycyrrhetinate, a silica, and Magnolia officinalis bark extract, wherein the phase B component is 5 wt % to 15 wt % of the solid cosmetic; and
a phase C component comprising Tanacetum annum essential oil, Cananga Odorata Genuina flower extract, Jasmine oil, and Chamomilla recutita extract, wherein the phase C component is 0.01 wt % to 1 wt % of the solid cosmetic;
wherein water and polyamides are less than 0.01 wt % of the solid cosmetic.

Embodiment 121 provides a method of making the solid cosmetic of any one of Embodiments 1-120, the method comprising:
combining the amino acid oil-gelling agent and one or more oils comprising the fatty acid ester component comprising a (C₅-C₁₅)alkyl ester of a (C₁₄-C₂₀)fatty acid to form a mixture;
heating the mixture to a temperature within and not exceeding a first temperature range of 145-160° C. sufficiently to substantially dissolve the amino acid oil-gelling agent in the one or more oils;
adding the phase B component comprising an antioxidant, a plant oil, an evergreen flower extract, a Hibiscus seed oil, a licorice extract or derivative thereof, a filler, a flowering tree bark extract, or a combination thereof to the mixture and heating the mixture within and not exceeding the first temperature range sufficiently to achieve a translucent mixture; and
transferring the translucent mixture to molds while the translucent mixture is within and not less than a second temperature range of 140-155° C., to form the solid cosmetic of any one of Embodiments 1-120.

Embodiment 122 provides the method of Embodiment 121, wherein the first temperature range is 150-158° C.

Embodiment 123 provides the method of any one of Embodiments 121-122, wherein the first temperature range is 154-156° C.

Embodiment 124 provides the method of any one of Embodiments 121-123, wherein the second temperature range is 145-153° C.

Embodiment 125 provides the method of any one of Embodiments 121-124, wherein the second temperature range is 147-149° C.

Embodiment 126 provides the method of any one of Embodiments 121-125, wherein the transferring of the translucent mixture to the molds while the translucent mixture is within and not less than a second temperature range comprises transferring the translucent mixture to the molds without any intermediary steps of storage, cooling below the second temperature range, and reheating.

Embodiment 127 provides the method of any one of Embodiments 121-126, wherein the transferring of the translucent mixture to the molds while the translucent mixture is within and not less than a second temperature range comprises transferring the translucent mixture to the molds before the translucent mixture cools below 145° C.

Embodiment 128 provides the method of any one of Embodiments 121-127, wherein the transferring of the translucent mixture to the molds while the translucent mixture is within and not less than a second temperature range comprises transferring the translucent mixture to the molds before the translucent mixture cools below 147 ° C.

Embodiment 129 provides the method of any one of Embodiments 121-128, further comprising adding the phase C component comprising a tansy oil, an Annonaceae flower oil, an Oleaceae flower oil, a chamomile extract, or a combination thereof to the translucent mixture prior to the transferring to the molds, and heating the mixture to within and not exceeding the second temperature range while agitating the mixture until it is again translucent prior to the transferring.

Embodiment 130 provides the method of Embodiment 129, further comprising heating the phase C component to within a third temperature range less than the second temperature range prior to combining the phase C component with the translucent mixture.

Embodiment 131 provides the method of Embodiment 130, wherein the third temperature range is 40-60° C.

Embodiment 132 provides the method of any one of Embodiments 130-131, wherein the third temperature range is 45-55° C.

Embodiment 133 provides the method of any one of Embodiments 129-132, wherein the transferring of the mixture to the molds is completed within 0.01-20 minutes of the achieving of the translucent mixture after the addition of the phase C component.

Embodiment 134 provides the method of any one of Embodiments 129-133, wherein the transferring of the mixture to the molds is completed within 0.01-10 minutes of the achieving of the translucent mixture after the addition of the phase C component.

Embodiment 135 provides the method of any one of Embodiments 129-134, wherein the transferring of the mixture to the molds is completed within 0.01-5 minutes of the achieving of the translucent mixture after the addition of the phase C component.

Embodiment 136 provides a method of making the solid cosmetic of any one of Embodiments 1-120, the method comprising:
combining the amino acid oil-gelling agent and one or more oils comprising the fatty acid ester component comprising a (C₅-C₁₅)alkyl ester of a (C₁₄-C₂₀)fatty acid to form a mixture; heating the mixture to a temperature within and not exceeding a first temperature range of 145-160° C. sufficiently to substantially dissolve the amino acid oil-gelling agent in the one or more oils;
adding the phase B component comprising an antioxidant, a plant oil, an evergreen flower extract, a Hibiscus seed oil, a licorice extract or derivative thereof, a filler, a flowering tree bark extract, or a combination thereof to the mixture and heating the mixture within and not exceeding the first temperature range sufficiently to achieve a translucent mixture;
heating the phase C component comprising a tansy oil, an Annonaceae flower oil, an Oleaceae flower oil, a chamomile extract, or a combination thereof to within a third temperature range less than the second temperature range, wherein the third temperature range is 40-60° C.;
adding the phase C component to the translucent mixture;
heating the mixture to within and not exceeding the second temperature range while agitating the mixture until it is again translucent; and
transferring the translucent mixture to molds while the translucent mixture is within and not less than a second temperature range of 140-155° C., to form the solid cosmetic of any one of Embodiments 1-120;
wherein
   the transferring of the translucent mixture to the molds while the translucent mixture is within and not less than a second temperature range comprises transferring the translucent mixture to the molds without any intermediary steps of storage, cooling, and reheating, and
   the transferring of the mixture to the molds is completed within 0.01-20 minutes of the achieving of the translucent mixture after the addition of the phase C component.

Embodiment 137 provides a method of making the solid cosmetic of any one of Embodiments 1-120, the method comprising:
combining the amino acid oil-gelling agent comprising dibutyl lauroyl glutamide and dibutyl ethylhexanoyl glutamide having a weight ratio of 1:1 to 5:1, one or more oils comprising ethylhexyl olivate, and one or more seed or vegetable oils comprising Jojoba oil, to form a mixture;
heating the mixture to a temperature within and not exceeding a first temperature range of 154-156° C. sufficiently to substantially dissolve the amino acid oil-gelling agent in the one or more oils;
adding the phase B component comprising tetrahexyldecyl ascorbate, Cocos Nucifera oil, Gardenia Tahitensis flowers extract, Hibiscus sabdariffa seed oil, stearyl glycyrrhetinate, a silica, and Magnolia officinalis bark extract to the mixture and heating the mixture within and not exceeding the first temperature range sufficiently to achieve a translucent mixture;
heating the phase C component comprising a Tanacetum annum essential oil, Cananga. Odorata Genuina flower extract, Jasmine oil, and Chamomilla recutita extract to within a third temperature range less than the second temperature range, wherein the third temperature range is 45-55° C.;
adding the phase C component to the translucent mixture;
heating the mixture to within and riot exceeding the second temperature range while agitating the mixture until it is again translucent; and
transferring the translucent mixture to molds while the translucent mixture is within and not less than a second temperature range of 147-149° C., to form the solid cosmetic of any one of Embodiments 1-120;
wherein
   the transferring of the translucent mixture to the molds while the translucent mixture is within and not less than a second temperature range comprises transferring the translucent mixture to the molds without any intermediary steps of storage, cooling below the second temperature range, and reheating,
   the transferring of the mixture to the molds is completed within 0.01-10 minutes of the achieving of the translucent mixture after the addition of the phase C component,
   the amino acid oil-gelling agent is 5 wt % to 15 wt % of the solid cosmetic,
   the fatty acid ester component is 35 wt % to 45 wt % of the solid cosmetic,
   the Jojoba oil is 30 wt % to 50 wt % of the solid cosmetic,
   the phase B component is 5 wt % to 15 wt % of the solid cosmetic, and
   the phase C component is 0.01 wt % to 1 wt % of the solid cosmetic.

Embodiment 138 provides a packaged product, comprising:
the solid cosmetic of any one of Embodiments 1-120; and
packaging that is resistant to the absorption of oils from the solid cosmetic.

Embodiment 139 provides the packaged product of Embodiment 138, wherein the packaging comprises paper comprising a coating that resists absorption of oils from the solid cosmetic into the paper.

Embodiment 140 provides the packaged product of Embodiment 139, wherein the coating is free of silicon, fluorocarbons, plastics, BPA, or a combination thereof, and/or wherein the paper is free of non-recycled materials.

Embodiment 141 provides the packaged product of any one of Embodiments 139-140, wherein the coating is free of silicon, fluorocarbons, plastics, and BPA, and wherein the paper is free of non-recycled materials.

Embodiment 142 provides the packaged product of any one of Embodiments 139-141, wherein the coating comprises a soy-based UV-cured coating that resists absorption of oils from the solid cosmetic into the paper.

Embodiment 143 provides a refillable skin care system comprising
the solid cosmetic of any one of Embodiments 1-120; and
a refillable container for storing the solid cosmetic.

Embodiment 144 provides the refillable skin care system of Embodiment 143, wherein the refillable container comprises a metal, a metal alloy, or a combination thereof

Embodiment 145 provides the refillable skin care system of any one of Embodiments 143-144, wherein the refillable container comprises steel.

Embodiment 146 provides the refillable skin care system of any one of Embodiments 143-145, wherein the refillable container comprises tin-coated steel.

Embodiment 147 provides the solid cosmetic, method of making the solid cosmetic, packed product, or refillable skin care system of any one or any combination of Embodiments 1-146 optionally configured such that all elements or options recited are available to use or select from.

What is claimed is:

1. A solid cosmetic comprising:
an amino acid oil-gelling agent; and
a fatty acid ester component comprising a (C₅-C₁₅)alkyl ester of a (C₁₄-C₂₀)fatty acid;
wherein the solid cosmetic is substantially free of water and polyamides.

2. The solid cosmetic of claim 1, wherein the solid cosmetic is transparent or translucent, and is free of suspended particles as measured by inspection with the naked eye.

3. The solid cosmetic of claim 1, wherein less than 0.01 wt % of the oil solid cosmetic is polyamides, and the solid cosmetic is less than 0.01 wt % water.

4. The solid cosmetic of claim 1, wherein the (C₅-C₁₅)alkyl ester of the (C₁₄-C₂₀)fatty acid is a (C₇-C₁₀)alkyl ester, and wherein the (C₁₄-C₂₀)fatty acid is a (C₁₆-C₁₈)fatty acid.

5. The solid cosmetic of claim 1, wherein the fatty acid ester component comprises a (C₅-C₁₅)alkyl ester of a (C₁₆)fatty acid and a (C₅-C₁₅)alkyl ester of a (C₁₈)fatty acid.

6. The solid cosmetic of claim 1, wherein the fatty acid ester component comprises ethylhexyl hexadecanoic acid, ethylhexyl palmitate, ethylhexyl linoleate, or a combination thereof.

7. The solid cosmetic of claim 1, wherein the fatty acid ester component is 20 wt % to 60 wt % of the solid cosmetic.

8. The solid cosmetic of claim 1, wherein the amino acid oil-gelling agent is 1 wt % to 30 wt % of the solid cosmetic.

9. The solid cosmetic of claim 1, wherein the amino acid oil-gelling agent comprises:
dibutyl lauroyl glutamide that is 1 wt % to 30 wt % of the solid cosmetic, and
dibutyl ethylhexanoyl glutamide that is 0.1 wt % to 10 wt % of the solid cosmetic;
wherein a weight ratio of the dibutyl lauroyl glutamide to the dibutyl ethylhexanoyl glutamide is 1:1 to 5:1.

10. The solid cosmetic of claim 1, further comprising one or more seed or vegetable oils that are 20 wt % to 60 wt % of the solid cosmetic, wherein the one or more seed or vegetable oils, the amino acid oil-gelling agent, and the fatty acid ester component, are together 70 wt % to 99 wt % of the solid cosmetic.

11. The solid cosmetic of claim 10, wherein the one or more seed or vegetable oils comprise Jojoba oil. 12 The solid cosmetic of claim 1, wherein the solid cosmetic comprises a triterpene that is 0.1 wt % to 10 wt % of the solid cosmetic.

13. The solid cosmetic of claim 1, further comprising a phase B component that is 1 wt % to 30 wt % of the solid cosmetic, the phase B component comprising
one or more antioxidants that are 0.01 wt % to 10 wt % of the solid cosmetic, or
one or more plant oils that are 1 wt % to 20 wt % of the solid cosmetic, or
one or more evergreen flower extracts that are 0.0001 wt % to 5 wt % of the solid cosmetic, or
a Hibiscus seed oil that is 0.0001 wt % to 5 wt % of the solid cosmetic, or
a licorice extract or derivative thereof that is 0.001 wt % to 5 wt % of the solid cosmetic, or
a filler that is 0.001 wt % to 5 wt % of the solid cosmetic, or
a flowering tree bark extract that is 0.0001 wt % to 5 wt % of the solid cosmetic, or
a combination thereof.

14. The solid cosmetic of claim 1, further comprising a phase C component that is 0.001 wt % to 5 wt % of the solid cosmetic, wherein the phase C component comprises
a tansy oil that is 0.0001 wt % to 5 wt % of the solid cosmetic, or
an Annonaceae flower oil that is 0.0001 wt % to 5 wt % of the solid cosmetic, or
an Oleaceae flower oil that is 0.0001 wt % to 5 wt % of the solid cosmetic, or
a chamomile extract that is 0.0001 wt % to 5 wt % of the solid cosmetic, or
a combination thereof.

15. A solid cosmetic comprising:
an amino acid oil-gelling agent comprising dibutyl lauroyl glutamide, dibutyl ethylhexanoyl glutamide, or a combination thereof, wherein the amino acid oil-gelling agent is 1 wt % to 30 wt % of the solid cosmetic;
a fatty acid ester component comprising a (C₅-C₁₅)alkyl ester of a (C₁₆)fatty acid and a (C₅-C₁₅)alkyl ester of a (C₁₈)fatty acid, wherein the fatty acid ester component is 20 wt % to 60 wt % of the solid cosmetic; and
one or more seed or vegetable oils that are 20 wt % to 60 wt % of the solid cosmetic;
wherein water and polyamides are less than 0.01 wt % of the solid cosmetic.

16. A solid cosmetic comprising:
an amino acid oil-gelling agent comprising dibutyl lauroyl glutamide and dibutyl ethylhexanoyl glutamide having a weight ratio of 1:1 to 5:1, wherein the amino acid oil-gelling agent is 5 wt % to 15 wt % of the solid cosmetic;
a fatty acid ester component comprising ethylhexyl olivate, wherein the -fatty acid ester component is 35 wt % to 45 wt % of the solid cosmetic;
one or more seed or vegetable oils comprising Jojoba oil, wherein the Jojoba oil is 30 wt % to 50 wt % of the solid cosmetic;
a phase B component comprising tetrahexyldecyl ascorbate, Cocos Nucifera oil, Gardenia Tahitensis flowers extract, Hibiscus sabdariffa seed oil, stearyl glycyrrhetinate, a silica, and Magnolia officinalis bark extract, wherein the phase B component is 5 wt % to 15 wt % of the solid cosmetic; and
a phase C component comprising Tanacetum annum essential oil, Cananga Odorata Genuina flower extract, Jasmine oil, and Chamomilla recutita extract, wherein the phase C component is 0.01 wt % to 1 wt % of the solid cosmetic;
wherein water and polyamides are less than 0.01 wt % of the solid cosmetic.

17. A method of making the solid cosmetic of claim 1, the method comprising:
combining the amino acid oil-gelling agent and one or more oils comprising the fatty acid ester component comprising a (C₅-C₁₅)alkyl ester of a (C₁₄-C₂₀)fatty acid to form a mixture;
heating the mixture to a temperature within and not exceeding a first temperature range of 145-160° C. sufficiently to substantially dissolve the amino acid oil-gelling agent in the one or more oils;
adding the phase B component comprising an antioxidant, a plant oil, an evergreen flower extract, a Hibiscus seed oil, a licorice extract or derivative thereof, a filler, a flowering tree bark extract, or a combination thereof to the mixture and heating the mixture within and not exceeding the first temperature range sufficiently to achieve a translucent mixture; and
transferring the translucent mixture to molds while the translucent mixture is within and not less than a second temperature range of 140-155° C., to form the solid cosmetic of claim 1.

18. The method of claim 17, further comprising adding the phase C component comprising a tansy oil, an Annonaceae flower oil, an Oleaceae flower oil, a chamomile extract, or a combination thereof to the translucent mixture prior to the transferring to the molds, and heating the mixture to within and not exceeding the second temperature range while agitating the mixture until it is again translucent prior to the transferring.

19. A packaged product, comprising:
the solid cosmetic of claim 1; and
packaging that is resistant to the absorption of oils from the solid cosmetic.

20. A refillable skin care system comprising
the solid cosmetic of claim 1; and
a refillable container for storing the solid cosmetic.
